(19) United States
(12) Patent Application Publication
(10) Pub. No.: US 2002/0015730 A1
(43) Pub. Date: Feb. 7, 2002

(54) Pharmaceutical formulations and method for making

(76) Inventors: Torsten Hoffmann, Radebeul (DE); Michael Pieroth, Weinbohla (DE); Gerhard Zessin, Halle/Saale (DE); Karl-Friedrich Landgraf, Dresden (DE)

Correspondence Address: Gabriel P. Katona L.L.P., 14th Floor, 708 Third Avenue, New York, NY, 10017, US

(21) Appl. No.: 09/793,936

(22) Filed: Feb. 27, 2001

Related U.S. Application Data
(60) Provisional application No. 60/187,962, filed on Mar. 9, 2000.

(51) Int. Cl. A61K 9/26; A61K 31/7016

(52) U.S. Cl. 424/470; 514/53

(57) ABSTRACT

The invention relates to an oral pharmaceutical formulation with variably adjustable release rate, which comprises one or more active ingredients, and one or more sucrose ester of a fatty acid as the sole release-controlling agent for said active ingredient wherein when the dosage form is a granule or a pellet, the formulation is made by melting the oral formulation, and granulating or pelletizing the melt.

### FIELD OF THE INVENTION

The present invention relates to new oral pharmaceutical formulations with variably adjustable release characteristics for the active ingredient, suitably in the form of granulates, pellets, tablets, film coated tablets, microtablets, sugar coated tablets, capsules or therapeutic systems, as well as to methods for their manufacture by melt granulation or melt pelletization.

### BACKGROUND

A reduced frequency of taking medicinal drugs and, in the ideal case, taking such drugs only once daily can play an important role in their use.

One tablet in the mornings or the evenings is usually taken more regularly than are several tablets spread over the day. In addition to the convenience, this improved patient compliance also has a positive effect on the healing process. In addition, the better compatibility of the active ingredient, which is frequently associated with a reduced frequency of taking it, benefits the patient. The latter is related to the need to maintain the effective plasma concentration for a longer time and to the mostly more uniform plasma levels, at which incompatible peak levels are largely avoided.

In exceptional cases, a single administration can already be realized by the kinetic or dynamic properties of an active ingredient, such as by a long elimination half-life. In most cases, however, effective plasma levels over 12 to 24 hours become possible only by pharmaceutical and technological measures, such as the delayed release of the active ingredient from the form in which it is administered.

The literature describes a series of solutions which in principle, depending on the chemical and physical properties of the active ingredient, have advantages or disadvantages (e.g. see the review article: Recent Trends and Progress in Sustained or Controlled Oral Delivery of Some Water Soluble Drugs, Drug Development and Industrial Pharmacy 21 (9), 1037-1070 (1998)).

The state of the art is given, for example, also in one of the newer textbooks of pharmaceutical technology (Voigt, R., Pharmazeutische Technologie (Pharmaceutical Technology), Ullstein Mosby Publishers 1993, page 293 ff.). According to this, the action of drugs can be prolonged by measures such as: varying the molecule, for example, by forming a salt or an ester, changing the active ingredient modification, the particle size, the choice of appropriate inert ingredients and the appropriate methods. Some exemplary possibilities are discussed below.

(a) Matrix Forms for Controlled Release of Drugs

These are characterized by an insoluble, possibly porous framework of indigestible fats, waxes, polymers or also inorganic matrix-forming materials. The active ingredient is incorporated into this framework and released by diffusion, erosion or matrix decomposition.

(b) Hydrocolloid Forms for Controlled Release of Drugs

The drug is incorporated in this case hydrocolloid matrices, such as cellulose derivatives. After the drug is ingested, a gel is formed by the digestion fluids. The active ingredient diffuses from the gel at a rate, which depends on the surface area and the gel viscosity.

(c) Coated (membrane-controlled) Forms for Controlled Release of Drugs

Active ingredient particles or drug forms are enveloped in these cases by a barrier. Diffusion through the diffusion barrier determines the rate of release of the active ingredient. Plasticizers or pore-forming agents can be added to increase the diffusion rate.

(d) Effect of the Specific Surface Area

For active ingredients having a low water solubility, there is generally a clear relationship between their rate of dissolution and their specific surface area. A defined particle size distribution and, and thus a particular specific surface area can be achieved by selective crystallization of the active ingredient, by screening or by grinding. The larger the particles, the smaller is the specific surface area and the slower is the release of active ingredient.

(e) Mixed Forms of Diffusion, Erosion and Dissolving Processes

Drug forms are known, for which the delayed release of the active ingredient is based on a combination of diffusion, erosion and dissolving processes.

Melt granulation represents a particularly interesting and, with respect to the release of active ingredient, very variably usable method. Melt granulation or thermoplastic granulation is a process, for which granulate bonding is brought about through the use of a low-melting component, as well as under the influence of thermal energy (Lüidemann, J.: APV Course 231 of Jun. 17 to 18, 1996).

A differentiation is made here between two sub-types. In the case of wet granulation, the process temperature is above the melting point of the binding component. The latter is present during the granulation as a liquid or semi-solid component. In melt granulation, drying is replaced by cooling.

Melt granulation is a sinter granulation, when the process temperature does not reach the melting point of the binding component. Only local melting at the surface of the particles takes place, so that the surfaces diffuse into one another (Voigt, R: Lehrbuch of pharmezeutischen Technologie (Textbook of Pharmaceutical Technology), Verlag Chemie, page 159 (1984)).

The low-melting component can be an active component or an inactive ingredient. For stability reasons, the melting points of the substances are generally above 35° C. The most frequently used materials have melting points ranging from 50° to 90° C. Known active ingredients, as fusible substances, are phenyl salicylate, ibuprofen, α-liponic acid and meprobamate. Water soluble, swellable and lipophilic substances are used as fusible inert ingredients. For example, Macrogol, Polyvidon and polymethacrylic acid derivatives are used as hydrophilic materials. Hydrocarbons (paraffins), waxes, fats and fatty acids are examples of inert lipophilic materials. (Flanders, P.; Dyer, G.A.; Jordan, D.; Drug Dev. Ind. Pharm. 13 (&), 1001-1022 (1987); Schaefer, T.; Holm, P.; Kristensen, H.G.; Drug Dev. Ind. Pharm. 16, 1249-1277 (1990); McTaggart, C.M. et al.; Int. J. Pharm. 19, 139-148 (1984); Kinget, R.; Kernel, R.; Acta Pharm. Technol. 31, 57 (1985)).

Melt granulation is usually carried out in fluidized bed granulators, centrifugal fluidized bed equipment or high-speed intensive mixers. The use especially of the latter has processing advantages, since a cost intensive air preparation can be omitted. Compared to conventional granulation methods with organic solvents. There are no expenses for explosion protection and solvent recovery compared to nonaqueous granulation. There are also no residual solvents in the product. There are no energy-consuming drying processes. The use of so-called one-reactor systems is preferred in such cases.

The process of melt granulation can in general be shown as follows:

| | |
|---|---|
| Mixing | Mixing |
| ↓ | ↓ |
| Addition of binder (solid | Heating |
| aggregate state) | ↓ |
| ↓ | |
| Heating | Addition of binder (solid |
| ↓ | aggregate state) |
| | ↓ |
| Granulation | Granulation |
| ↓ | ↓ |
| Cooling | Cooling |
| optionally classifying | ↓ |
| | optionally classifying |

Fusible binders can be added in the solid or liquid state, that is, in the molten state.

For solid addition, the fusible material is melted during the process. For this reason, this method is also referred to as the melting method.

For the latter method, either the solid components are transferred to the reactor first and the liquid binder is added or, corresponding to the so-called fusion method, the liquid binder is added to the reactor and the solid materials are stirred in. For this purpose, heating is carried out before the addition of the binder.

In the case of intensive mixers, energy can be supplied in various ways:

mechanical energy by mixing tools and choppers;

contact heat by way of a heating jacket;

radiation energy by IR or microwave;

hot air introduction into the product bed.

A large number of methods for producing such formulations are also known from the patent literature. Formulations with a controlled release, which can be produced by way of melt granulation, are described, for example, in German patent No. 2,426,812, European patents 351,580; 654,263; 672,416; and 729,751 and in WO 93/18753. The last one describes a process, in which water-insoluble, hydrophobic, wax-like substances are added at a later time in the production process to the prepared pellets at a temperature, at which these substances melt and lead to a coating of these substances. This process is referred to as “hot-melt coating”.

On the assumption that all of the starting materials, participating in the process are thermally stable under the existing process conditions, melt granulation is an interesting alternative to other granulation methods, such as granulating, for example, with organic solvents or granulating with water.

Melt pelletizing represents a special form of carrying out the process, for which the granulate particles are produced with a largely uniform size and rounded shape.

In spite of the large number of known non-active excipients, which can be melted, only a few such materials with graded HLB values (hydrophilic-lipophilic balance values) are described, which are particularly suitable for melt granulation processes or melt pelletization processes.

Representatives of the few inert ingredients with graded HLB values are hydrogenated edible fats, which are available under the trade name of Gelucires, or the sorbitol esters of fatty acids, which are known for example, as Span. However, these also do not cover the broad HLB range from 1 to 16.

With the classical, fusible inert ingredients, the release rates can be varied only by the retarding agent selected or by the amount of this agent. Frequently, a binder can be processed only in combination with a different fusible binder, such as polyethylene glycol, since its granulate-forming alone is inadequate. These binders also require the addition of lubricants or mold release agents. Some have a wax-like consistency. In the case of the known methods of melt granulation, the resulting, solidified granulates must frequently be subjected to an expensive screening process to comminute the product.

When preparing controlled release compositions by a coating procedure, destruction of the film coating is frequently observed during pressing because of the partially brittle, but also relatively thin film coatings, unless such a destruction is counteracted with a relatively large amount of external phase. When the film coating is destroyed, the release rate of active ingredient from the tablets is increased. This means that the release of active ingredient from these tablets mostly depends on the pressing force. Frequently, in the case of this method, the release of active ingredient is adjusted by the amount sprayed on during the manufacturing process. Depending on the film formation and the porosity, the release rate of active ingredient may change during storage, for example, due to post-curing.

### BRIEF DESCRIPTION OF THE DRAWING

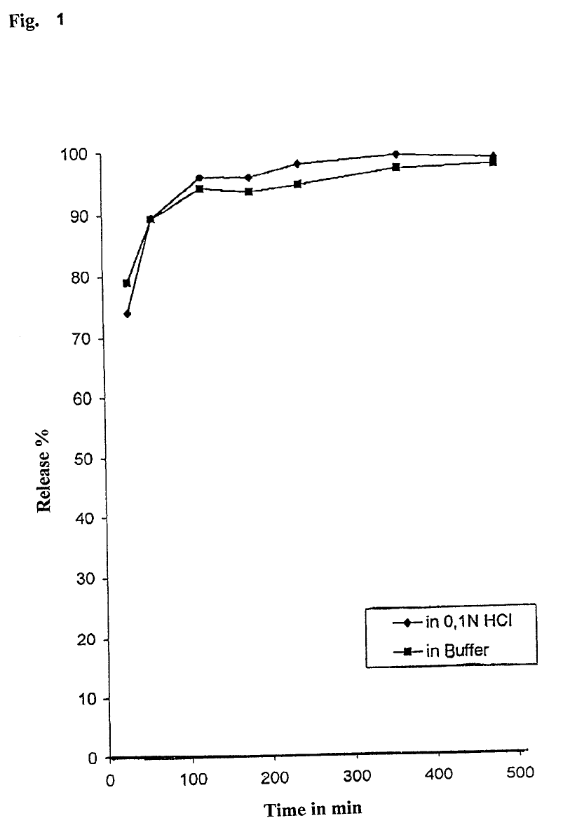

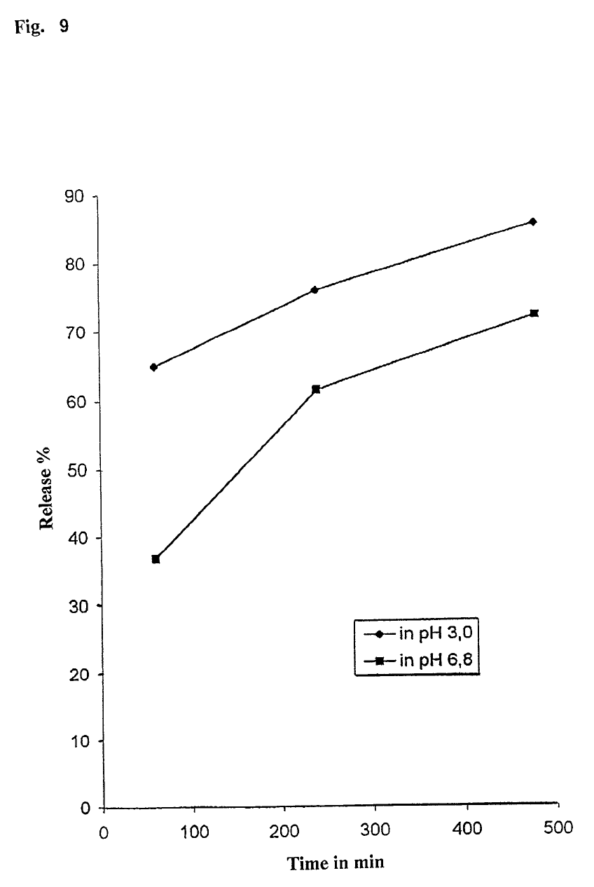

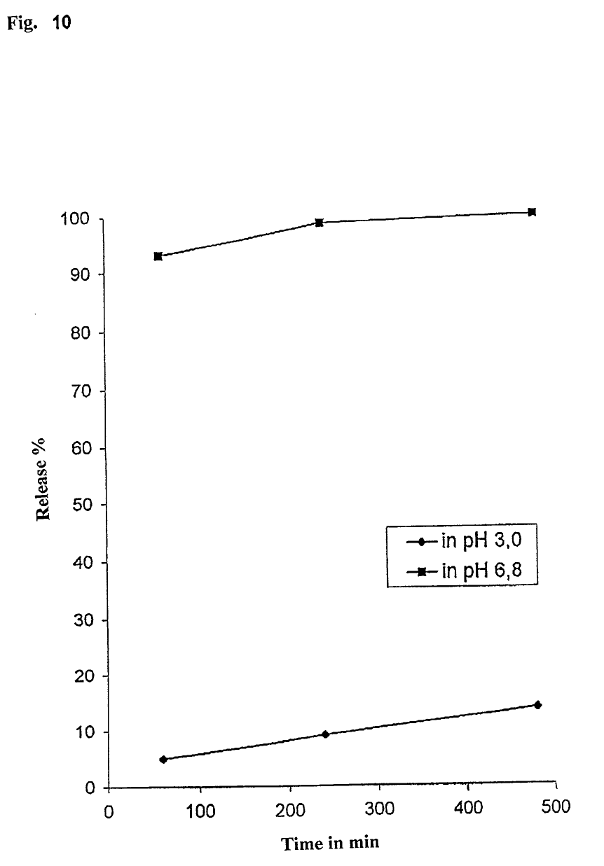

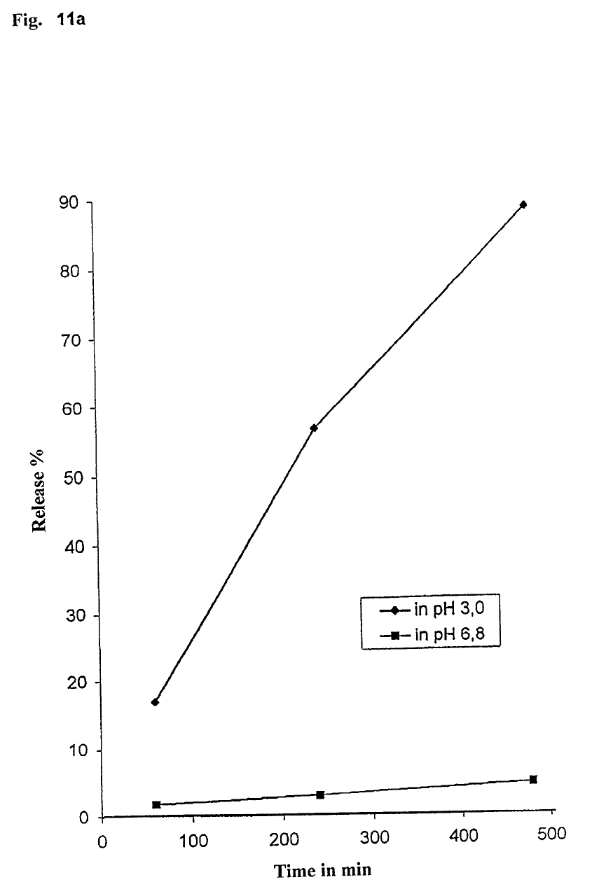

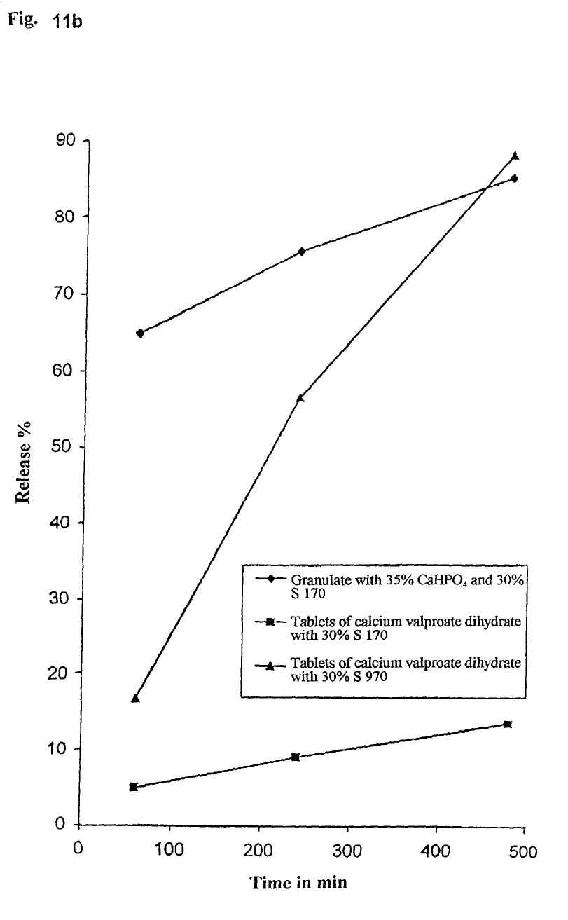

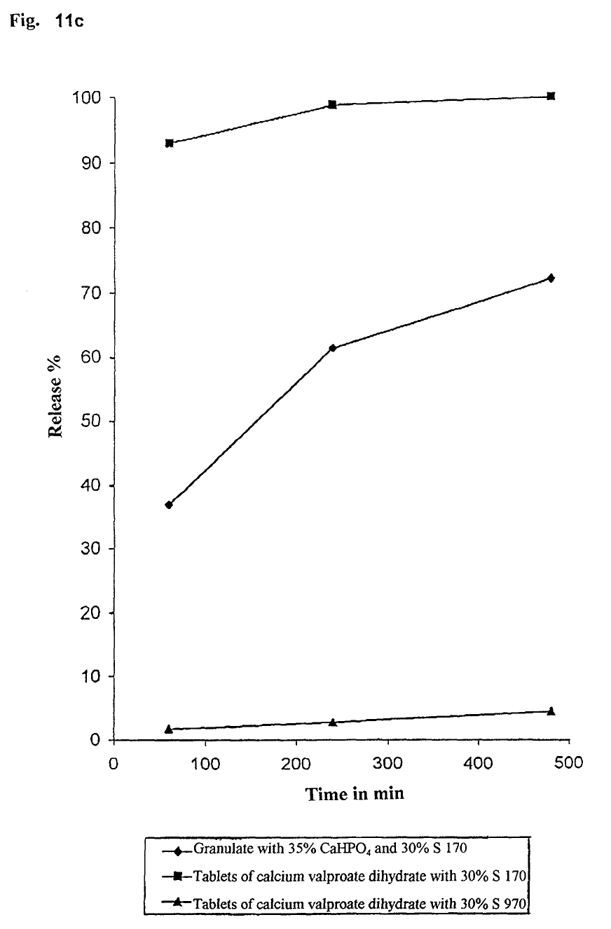

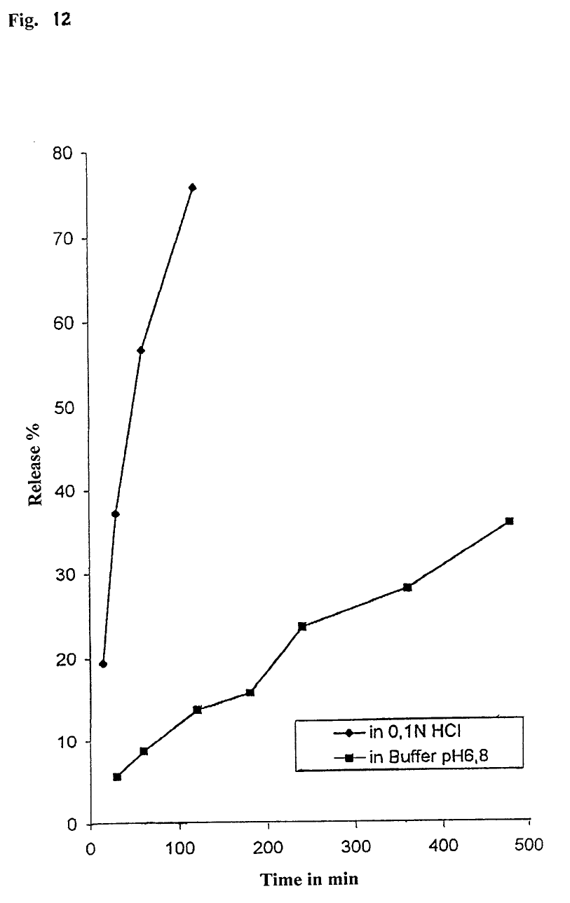

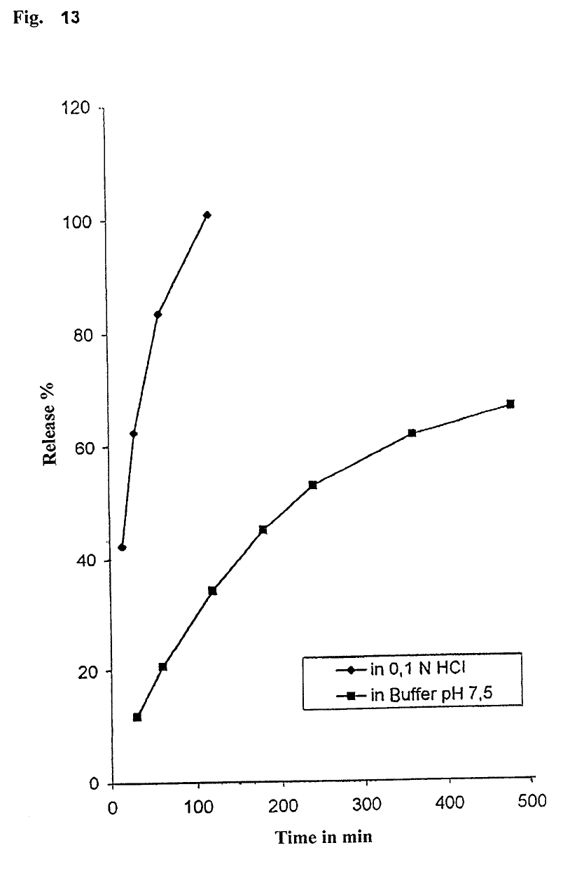

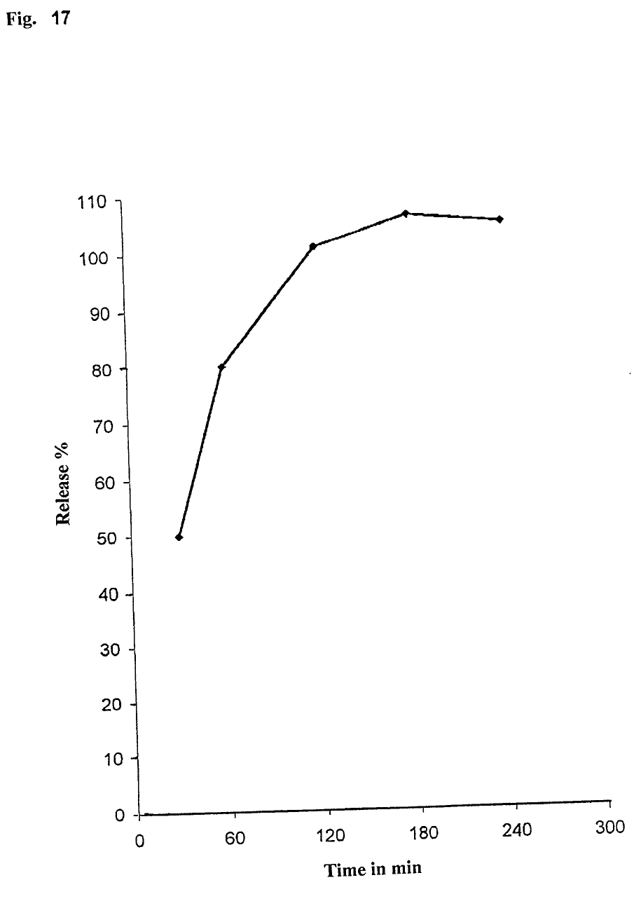

The invention is illustrated through the Examples and the appended drawing in which FIGS. 1-21b illustrate properties of compositions as prepared by the Examples.

### DESCRIPTION OF THE INVENTION

It is therefore an objective of the present invention to make oral pharmaceutical formulations available with a variable, adjustable release behavior, which can range from rapid to retarded. The release rate of the active ingredient of the dosage forms that are modified or retarded, is possible to produce non-disintegrating drug forms (so-called “single units”) as well as suitably rapidly disintegrating and modified or retarded drug forms (so-called “multiple unit forms”) from the granulates.

It is a further object of the present invention to provide methods for producing such retard or slow release formulations especially by melt granulation or melt pelletization.

According to the present invention, new oral pharmaceutical formulations with variably adjustable release behavior are provided which, in addition to one or more active ingredients, contain one or more sucrose esters of fatty acids as the sole release-controlling agent. The new pharmaceutical formulations are dosage forms, which release at various rates from immediate to controlled release.

As used throughout the disclosure and the claims, any reference to any active ingredient is meant also to include optionally more than one active ingredient, and reference to a sucrose ester of a fatty acid also includes optionally more than one sucrose ester of a fatty acid.

The pharmaceutical formulations of the present invention can be administered in the form of granules, pellets, tablets, film-coated tablets, microtablets, sugar-coated tablets and capsules and as therapeutic systems.

Surprisingly, sucrose esters of fatty acids are able to control the release of active ingredients in the desired manner and, moreover, to improve the technological properties during the preparation of the formulations of the invention by melt granulation or melt pelletization.

Sucrose esters of fatty acids are also suitable for granulating the active ingredient without the addition of other inert materials. By these means, a gross reduction in weight is possible in comparison to other methods, in which several fusible retarding agents or binders have to be used. At the same time, sucrose esters of fatty acids, particularly stearates with a low HLB value, such as from about 1 to about 16 can be suitably used as lubricants and as mold release agents.

Sucrose esters of fatty acids are nonionic surfactants, which are mono-, di-, tri-and polyesters of sucrose as the hydrophilic component and saturated or unsaturated fatty acids as the lipophilic component. By varying the degree of esterification and the nature of the fatty acids, sucrose esters of fatty acids can be produced with different HLB values, which have an effect on the biopharmaceutical properties, especially the release of active ingredient, the stability of the pharmaceutical formulation produced and its technological behavior. They are nontoxic, biodegradable, tasteless and odorless and have a long shelf life. The sucrose esters of fatty acids with a melting point higher than 30° C. are solid at room temperature and have an BLB value of from 1 to 16.

Sucrose esters of fatty acids are also sold under the name, for example, of sugar esters or sucrose esters by Mitsubishi (under the trade name of Ryoto), Gattefosse, or Sisterna and others.

Sucrose esters of fatty acids known from the literature are, for example, those of U.S. Pat. No. 4,844,067 used to improve the surface of silk fibers, and those of WO 93/17667 as taste improvers in pharmaceutical preparations.

Their main use is in the food industry. For example, sucrose esters of fatty acids are used to improve the mixing of chewing gum compositions, to counteract demixing and denaturing of finished beverages, for refining sugar, in condensed milk and in coffee creamers.

Sucrose esters of fatty acids are used for the production of wheat flour products, for example, as stabilizers to improve the texture and to avoid baking on and sticking on. In milk products they are used to stabilize emulsions and to avoid proteins and degradation, sucrose esters of fatty acids improve the crystallization behavior, and are effective emulsifiers and lower the viscosity during the production all fats and oils.

In U.S. Pat. Nos. 3,896,238; 4,150,114; and 4,046,886; the use of sucrose esters of fatty acids in combination with alkyl sulfoxide or phosphorus oxides in pharmaceutical compositions is disclosed for improving the penetration of the active substance through the skin. Sucrose monooctonate, monolaurate, monopalmitate and monostearate, as well as diesters and triesters of these compounds are named as special sucrose esters of fatty acids. In Japanese patent No. 8,175,437, the use of sucrose esters of fatty acids with an HLB value of 1 to 5 is disclosed as a base for suppositories.

In WO 88/06880, the use of sucrose esters of fatty acids in topical applications is disclosed, mixtures of mono-and dialkyl sucrose esters with an HLB value of 8 to 16 being used to improve the penetration through the skin. Preferably, sucrose cocoate, sucrose ricinoleate, sucrose laurate and sucrose stearate are used for that purpose.

Sucrose esters of fatty acids are also used, particularly, in cosmetic products (French patent No. 2,421,605, and Japanese patents Nos. 8,124,034 and 8,155, 306).

In German patent No. 4,003,844, pharmaceutical compositions are described which, in addition to the active ingredient, cyclosporin, contain a sucrose monoester of a fatty acid and a diluent or carrier. These compositions enable the cyclosporin dosage level, required for achieving an effective therapy, to be reduced and, thus, lead to a reduction in undesirable side effects. As sucrose monoester of a fatty acid, monoesters of C₆₋₁₄ and C₈₋₁₈ fatty acids are particularly suitable for that purpose.

In WO 93/00093, a new controlled release formulation for Diltiazem in the form of spheroids is disclosed, which is composed of the active ingredient, a wetting agent and a polymer coating for controlling the release. Sucrose esters of fatty acids are used as a wetting agent. The actual retardation of active release takes place by a polymer. Moreover, the wetting agent is processed with the active ingredients by extrusion or by granulation with organic solvents. The extrudates are coated with conventional polymers. Sucrose or xylose esters of C₁₂₋₂₀ fatty acids, for example, are named as wetting agents.

In German patent No. 19,840,152, a retard formulation is disclosed, which contains calcium valproate, at least one acrylic polymer and at least one sugar ester, wherein the desired retarding effect being achieved by the acrylic polymer that is used. It is shown that the sugar ester, by itself, does not have any meaningful release retarding effect.

The suitability of sucrose esters of fatty acids to be the sole release-controlling agent in the pharmaceutical formulations of the present invention was all the more surprising, since these sucrose esters of fatty acids, on the one hand, have already been known for a long time per se, and now in accordance with the present invention can be employed in a simple manner in oral pharmaceutical formulations, with a variably adjustable release behavior.

The sucrose esters of fatty acids, used pursuant to the present invention, are esters of sucrose with saturated or unsaturated fatty acids or mixtures thereof Particularly suitable are C₁₂₋₂₂ fatty acids. Sucrose stearates, sucrose palmitates, sucrose laurates, sucrose behenates and sucrose oleates, with an HLB value of about 1 to about 16, are suitably used. The melting point or melting range of the sucrose esters of fatty acids, which are used pursuant to the invention, lies between about 30° C. and about 200° C. Suitably, sucrose esters of fatty acids with a melting point or melting range of from about 40° C. to about 150° C. are used.

An essential advantage of the present invention is that the desired release behavior of the new pharmaceutical formulations can be controlled by the type and proportion of the sucrose fatty acid ester or esters used and by the parameters of the manufacturing process. Selection of an appropriate ester or combination of esters, and suitable processing parameters can be determined as the basis of guidelines disclosed herein and by routine experimentation.

Sucrose esters of fatty acids with a low HLB value are suitable for achieving a retarded release. Sucrose esters of fatty acids with a high HLB value are suitable for a more rapid or modified release behavior.

In the pharmaceutical formulations of the present invention, the sucrose esters of fatty acids can be used in amounts of from about 1% to about 95% by weight, based on the amount to be granulated (inner phase) in the formulation. More suitably, an amount of about 5% to about 50% by weight is used. Aside from sucrose esters of fatty acids, the active ingredient or mixtures of the active ingredient can also contain one or more inert excipients, such as are conventionally used in pharmaceutical preparations in the inner phase.

In further embodiments of the invention granules or pellets, which may or not contain sucrose esters of fatty acids in the granulate, can be coated instead with sucrose esters of fatty acids. The proportion of sucrose esters of fatty acids in the coating is from about 1% to about 60% by weight and suitably from about 3% to about 20% by weight, based on the coated form of the drug.

The sucrose esters of fatty acids can be used by themselves or optionally also in combination with other fusible inert ingredients. In some cases, the addition of one or more inert materials, such as plasticizers, can be of advantage for the process. A further modification of the release of active ingredient is possible by way of embedding suitably during the melt granulating or melt pelletizing process, a so-called pore-forming agent, an inert material with certain properties, such as having a characteristic solubility or swellability.

As active ingredients, the inventive, oral pharmaceutical formulations can contain compounds, the solubility of which in water ranges from good to practically insoluble.

For example, active ingredients of the following indication groups were found to be suitable for this purpose, analeptic agents, antihypoxemic agents (such as caffeine), analgesics, antirheumatic agents (such as diclofenac, morphine, tramadol, tilidin, flupirtin), antiallergic agents (such as azelastin, pseudoephedrine), antiarrhythmic agents (such as quinidine, disopyramide, diltiazem, verapamil), antidementia agents (nootropic agents) (such as piracetam, nicergolin, xantino nicotinate, pentifyllin, vincamin), antidiabetic agents (such as glibenclamide), antiemetic agents, antivertiginous agents (such as betahistin dimesilate, dimenhydrinate), antiepilieptic agents (such as carbamazepine, valproic acid, calcium valproate dihydrate, retigabine), antihypertensive agents (such as talinolol, fosinopril, doxazosin, metoprolol, nifedipine), antihypotensive agents (such as norfenefrin-HCl, dihydroergotamine mesilate), broncholytic agents, antiasthmatic agents (such as salbutamol, terbutalin sulfate, theophyllin), diuretics (such as furosemide, piretanide), circulation promoters (such as buflomedil, naftidrofuryl, pentoxifyllin), hypnotic agents, cardiac agents (such as trinitroglycerin, isosorbid mononitrate, isosorbid dinitrate, molsidomin), sedatives, lipid-lowering agents (such as bezafibrate, fenofibrate, xantinol), antimigraine preparations (such as sumatriptan), muscle relaxants, anti-Parkinson agents and other agents against extrapyramidal disorders (such as levodopa, benserazide, carbi-dopa), psycho-pharmaceuticals (such as amitriptylin-HCl, venlafaxin-HCl, thioridazin-HCl, lithium carbonate, lithium acetate), or their pharmaceutically acceptable salts.

The pharmaceutical formulations of the present invention can suitably contain flupirtin, tramadol, nifedipine, carbamazepine, calcium valproate or retigabine.

Pursuant to the present invention, the pharmaceutical formulations of the invention can be suitably prepared by melt granulation or melt pelletization. For this purpose, for example, the mixture of active ingredient and one or more sucrose esters of fatty acids is heated with stirring in a high-speed mixer, optionally together with other inert materials. The heating can be accomplished by a heating jacket, with microwave, by radiation energy or by supplying energy by stirring. Granulation commences when the melting temperature of the sucrose ester of the fatty acids used in the mixture is reached or when the surface of the mixture softens or commences to melt. Because of the agglomeration that commences and the increase in friction associated therewith, the power increases that is taken up by the stirrer motor. As a rule, the granulation is terminated when the power uptake commences to rise exponentially. After that, the warm melt granulate is either discharged from the mixture and cooled in thin layers at room temperature or cooled with suitable cooling means (such as a cooling jacket) in the mixer, possibly with stirring. Pursuant to the invention, it is also possible to add the sucrose esters of the fatty acids in the molten state.

Surprisingly, a very narrow distribution of granulate sizes is achieved during this process. Depending on the manner, in which the process is carried out, the granulate or pellet particles have an almost rounded and smooth surface.

Likewise, it is possible to use other equipment, which can be heated, such as a fluidized bed granulator, or a rotor granulator.

The granules, so produced, can optionally be classified through a screen, possibly mixed with inert ingredients of the outer phase and, for example, pressed into tablets, or filled into capsules.

The customary pharmaceutical disintegrants or disintegrating agents, fillers, mold release agents or the like are used as inert materials of the outer phase. Usually, the use of mold release agents can be omitted when sucrose stearates of low HLB value are used, since sucrose stearates with a low HLB themselves also represent good mold release properties.

Accordingly, depending on the pharmaceutical, technological objective, rapidly releasing formulations and formulations, the release from which is modified to retarded (multiple units or single units), can be produced.

It was moreover surprisingly found that the sucrose esters of fatty acids are suitable as inert ingredients for hot melt coating. For this purpose, an amount of sucrose esters of fatty acids of the same or of a different type is added once again to a melt granulate, which has already been produced and solidified and the mixture is heated once again above the melting point or the softening temperature of the sucrose ester of the fatty acids added. The sucrose ester of the fatty acids is coated over the melt granulate at the same time. The coating process can also take place in the presence of a plasticizer. Likewise, granulates, which are free of sucrose esters of fatty acids or pure active ingredients can be coated in the manner described.

The advantage of this method is that, on the one hand, a sufficient control of the release, particularly a retardation, can be attained already by coating with smaller amounts of the sucrose esters of fatty acids. On the other hand, the surface of the granules or pellets, so prepared is smoothened.

A further advantage is that by this method coatings, which are resistant to gastric juices, can be produced in a simple manner. Thus, the possibility exists that the release of active ingredient in the acid range of the pH can be greatly retarded because the sucrose ester of fatty acids is practically insoluble in aqueous and acidic media.

Powder coating represents a special form of hot melt coating. On the one hand, the readily flowable sucrose esters of fatty acids are added with the help of a suitable powder feeder, and on the other hand, a plasticizer, such as triethyl citrate is added to the starting materials. This method is distinguished by large cost and time savings, since drying processes, such as those employed in conventional aqueous methods, are not required. In particular, the so prepared pharmaceutical formulations are suitable for water-sensitive active ingredients, such as Na valproate.

The following examples further explain the present invention in greater detail.

### EXAMPLE 1

Tramadol Hydrochloride with 50% Sucrose Stearate with an HLB of 1
Formulation:

| | |
|---|---|
| Substance | Amount |
| Tramadol hydrochloride | 400 g |
| Sucrose stearate S-170 | 400 g |

Parameters:

| | |
|---|---|
| Amount formulated | 800 g |
| impeller speed | 700 rpm |
| Chopper speed | 3000 rpm |
| Heater jacket temp | 55.0° C. |

The starting materials are heated with stirring in an high shear mixer of the GP1 type of firm Aeromatic-Fielder at the appropriate jacket temperature. The granulation commences when the product reaches a particular temperature. When the increase in the power uptake is reached and there is a sudden increase in the product temperature, the granulation is discontinued and the product is discharged, screened at a mesh width of 1.4 mm and cooled to room temperature.

Evaluation: Active Ingredient Release

| | | | | | | | |
|---|---|---|---|---|---|---|---|
| Time in mm | 30 | 60 | 120 | 180 | 240 | 360 | 480 |
| Release in % | 74.03 | 89.40 | 95.75 | 95.57 | 97.61 | 98.58 | 97.87 |
| in 0.1 NHCl | | | | | | | |
| In buffer of pH 6.8 | 78.99 | 89.29 | 93.99 | 93.37 | 94.26 | 96.5 | 96.88 |

Active Ingredient Release: see FIG. 1

### EXAMPLE 2

Flupirtin maleate with 30% sucrose stearate of HLB 1

| | |
|---|---|
| Substance | Amount |

Formulation:

| | |
|---|---|
| Flupirtin maleate | 240.0 g |
| Sucrose stearate S-170 | 102.9 g |

Parameters:

| | |
|---|---|
| Amount of Formulation | 342.9 g |
| Impeller speed | 700 rpm |
| Chopper speed | 3000 rpm |
| Heater jacket temp. | 61.2° C. |

Produced as in Example 1

### EXAMPLE 3

Nifedipine with 30% sucrose stearate of HLB 1

| | |
|---|---|
| Substance | Amount |

Formulation:

| | |
|---|---|
| Nifedipine | 560 g |
| Sucrose stearate S-170 | 240 g |

Parameters:

| | |
|---|---|
| Amount of Formulation | 800 g |
| Stirrer speed | 700 rpm |
| Chopper speed | 3000 rpm |
| Mantle temperature | 58° C. |

Produced as in Example 1
Evaluation: Active Ingredient Release

| Time in hours | 1 | 2 | 4 | 6 | 8 | 24 |
|---|---|---|---|---|---|---|
| Release in % | 2.14 | 3.76 | 5.84 | 8.42 | 10.72 | 25.91 |
| in purified | | | | | | |
| water/1.25% SDS | | | | | | |

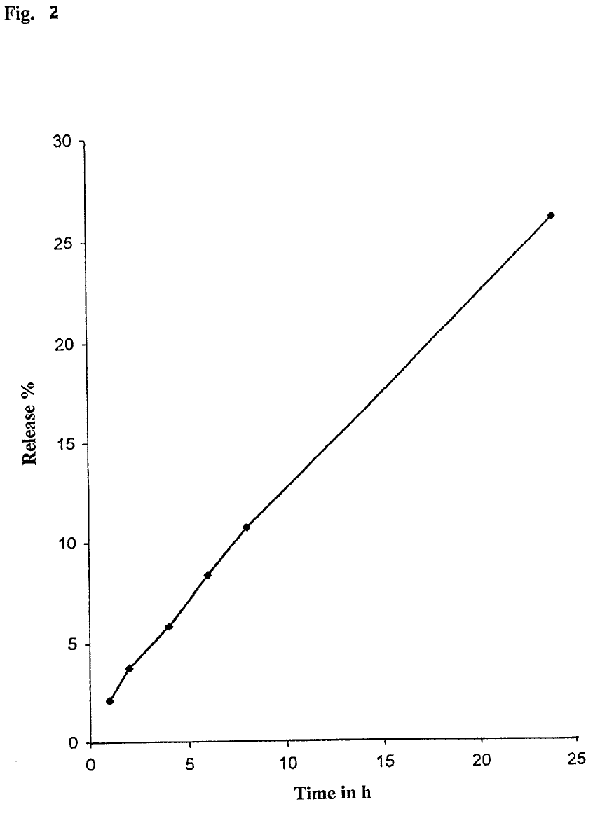

Active Ingredient Release: see FIG. 2

### EXAMPLE 4

Nifedipine with 30% sucrose palmitate of HLB 1

| | |
|---|---|
| Substance | Amount |

Formulation:

| | |
|---|---|
| Nifedipine | 560 g |
| Sucrose palmitate P-170 | 240 g |
| Parameters: | |
| Amount of formulation | 800 g |
| Impeller speed | 700 rpm |
| Chopper speed | 3000 rpm |
| Heater jacket temp. | 52° C. |

Preparation as in Example 1
Evaluation: Active Ingredient Release

| Time in hours | 1 | 2 | 4 | 6 | 8 | 24 |
|---|---|---|---|---|---|---|
| Release in % | 4.08 | 7.32 | 11.5 | 16.65 | 21.71 | 49.04 |
| in purified | | | | | | |
| water/1.25% SDS | | | | | | |

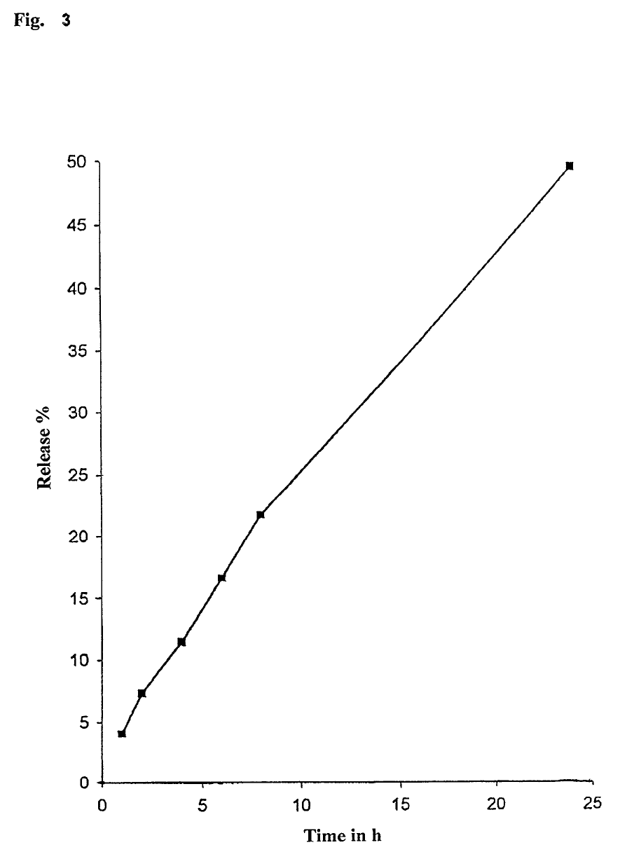

For Active Ingredient Release, see FIG. 3

### EXAMPLE 5

Tablets from melt granulate of nifedipine and 30% sucrose
stearate of HLB 5

| | |
|---|---|
| Substance | Amount |

Formulation:

| | |
|---|---|
| Nifedipine | 560 g |
| Sucrose stearate P-170 | 240 g |

Parameters for granulating:

| | |
|---|---|
| Amount of formulation | 800 g |
| Impeller speed | 700 rpm |
| Chopper speed | 3000 rpm |
| Heater jacket temp. | 70° C. |

Produced as in Example 1

Parameters for tableting:
Subsequently, the granulate was pressed with a circular tableting
tool with a diameter of 6 mm with medium convexity into tablets
with a gross weight of 71.4 mg.
Evaluation: Active Ingredient Release

| Time in min | 30 | 60 | 120 | 180 | 240 |
|---|---|---|---|---|---|
| Release in % in purified | 19.85 | 42.44 | 78.30 | 96.61 | 102.88 |
| water/1.25% SDS | | | | | |

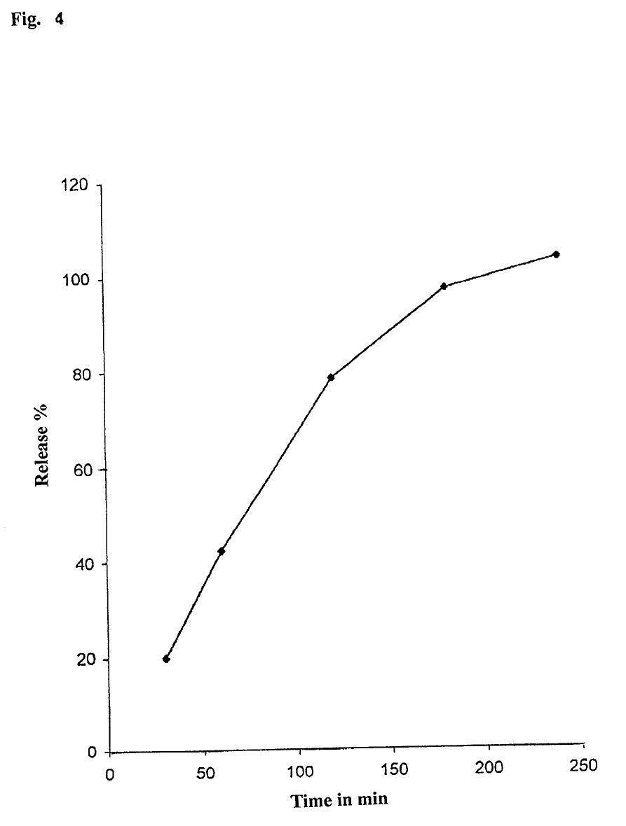

Active Ingredient Release: see FIG. 4

### EXAMPLE 6

Tablets from melt granulate of nifedipine with 50% sucrose
stearate of HLB 9 and 2.5% sucrose stearate of HLB 1

| | |
|---|---|
| Substance | Amount |

Formulation:

| | |
|---|---|
| Nifedipine | 400 g |
| Sucrose stearate S-970 | 380 g |
| Sucrose stearate S-170 | 20 g |

Parameters for granulating:

| | |
|---|---|
| Amount of formulation | 800 g |
| Impeller speed | 700 rpm |
| Chopper speed | 3000 rpm |
| Heater jacket temp. | 65° C. |

Produced according to Example 1
Parameters for tableting:
Subsequently, the granulate was pressed with a circular tableting
tool with a diameter of 6 mm with medium convexity into tablets
with a gross weight of 100 mg.
Evaluation of Active Ingredients Release:

| Time in min | 30 | 60 | 120 | 180 | 240 |
|---|---|---|---|---|---|
| Release in % | 20.10 | 40.37 | 73.26 | 94.14 | 102.93 |
| in purified water/1.25% SDS | | | | | |

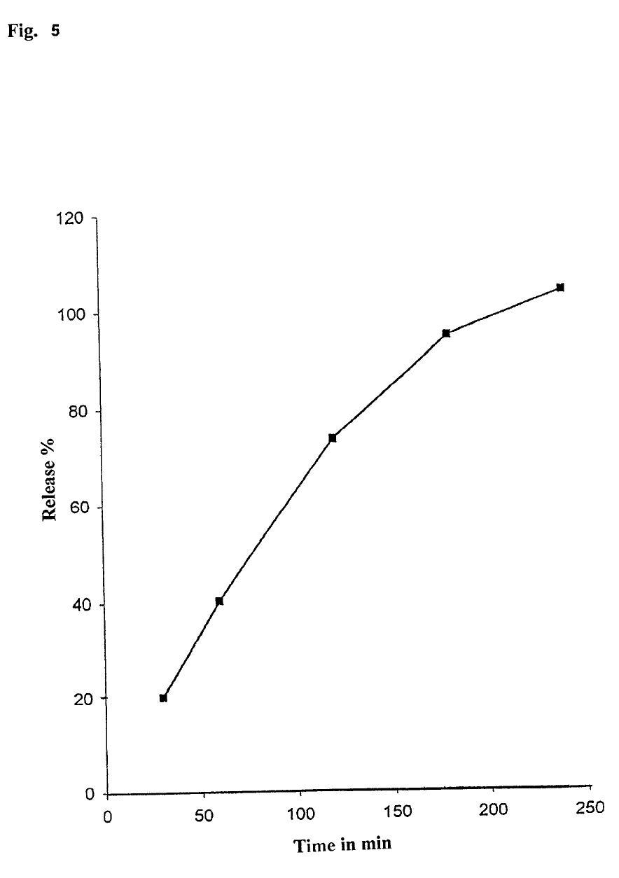

Active Ingredient Release: see FIG. 5

### EXAMPLE 7

Carbamazepine with 10% sucrose stearate of HLB 1

| Formulation: | |
|---|---|
| Substance | Amount |
| Carbamazepine | 720 g |
| Sucrose stearate S-170 | 80 g |
| Parameters: | |
| Amount of formulation | 800 g |
| Impeller speed | 700 rpm |
| Chopper speed | 3000 rpm |
| Heater jacket temp. | 55° C. |

Prepared according to Example 1

Evaluation: Active Ingredient Release

| | | | | | | | |
|---|---|---|---|---|---|---|---|
| Time in min | 30 | 60 | 120 | 180 | 240 | 360 | 480 |
| Release in % | 10.68 | 20.06 | 38.08 | 51.45 | 62.47 | 73.89 | 81.58 |
| in modified | | | | | | | |
| intestinal juice | | | | | | | |

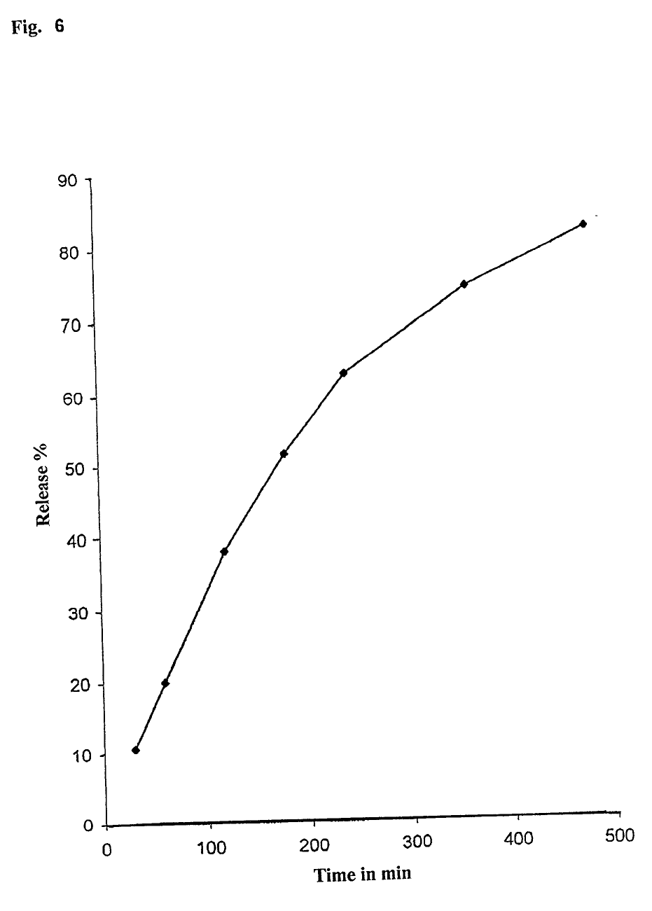

Active Ingredient Release: see FIG. 6

### EXAMPLE 8

Carbamazepine with 30% sucrose stearate of HLB 9

| Formulation: | |
|---|---|
| Substance | Amount |
| Carbamazepine | 560 g |
| Sucrose stearate S-970 | 240 g |
| Parameters: | |
| Amount of formulation | 800 g |
| Impeller speed | 700 rpm |
| Chopper speed | 3000 rpm |
| Heater jacket temp. | 68° C. |

Prepared according to Example 1

Evaluation: Active Ingredient Release

| | | | | | | | |
|---|---|---|---|---|---|---|---|
| Time in min | 30 | 60 | 120 | 180 | 240 | 360 | 480 |
| Release in % | 26.09 | 42.27 | 62.65 | 80.58 | 87.38 | 96.56 | 100.84 |
| in modified | | | | | | | |
| intestinal | | | | | | | |
| juice | | | | | | | |

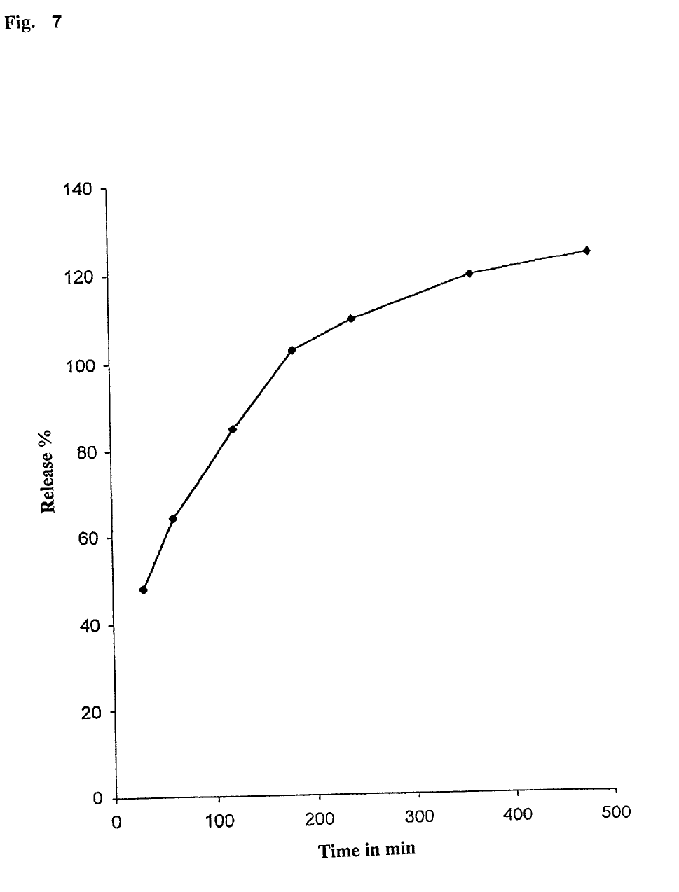

Active Ingredient Release: see FIG. 7

### EXAMPLE 9

Carbamazepine with 50% sucrose behenate of
HLB 3 and 2.5% triethyl citrate

| Formulation: | |
|---|---|
| Substance | Amount |
| Carbamazepine | 400 g |
| Sucrose behenate B-370 | 380 g |
| Triethyl citrate | 20 g |
| Parameters: | |
| Amount of formulation | 800 g |
| Impeller speed | 700 rpm |
| Chopper speed | 3000 rpm |
| Heater jacket temp. | 50° C. |

The starting materials, carbamazepine and triethyl citrate, are mixed in an
intensive mixer of the GP1 type of firm Aeromatic-Fielder.
After a mixing time of 1 minute, sucrose behenate B-370 is added and
the mixture is heated with stirring at a heater jacket temperature of
50.0° C. When a particular product temperature is reached, at
which an increase in power may be noted, the granulate is added to
a screen with a mesh width of 1.4 mm and cooled to room temperature.

### EXAMPLE 10

Tablets of a melt granulate of carbamazepine
with 30% sucrose stearate of HLB 9

| Formulation: | |
|---|---|
| Substance | Amount |
| Carbamazepine | 560 g |
| Sucrose stearate S-970 | 240 g |
| Parameters for granulating: | |
| Amount of formulation | 800 g |
| Impeller speed | 700 rpm |
| Chopper speed | 3000 rpm |
| Heater jacket temp. | 68° C. |

Prepared according to Example 1
Parameters for tableting:
The granulate is pressed without further additions with a round
tableting tool with a diameter of 13 mm into flat tablets
with a gross weight of 571 mg and a crush strength of 25 N.

Evaluation: Release of Active Ingredient

| | | | | | | | |
|---|---|---|---|---|---|---|---|
| Time in min | 30 | 60 | 120 | 180 | 240 | 360 | 480 |
| Release in % in | 5.36 | 8.04 | 13.78 | 17.89 | 21.01 | 27.31 | 32.08 |
| modified | | | | | | | |
| intestinal | | | | | | | |
| juice | | | | | | | |

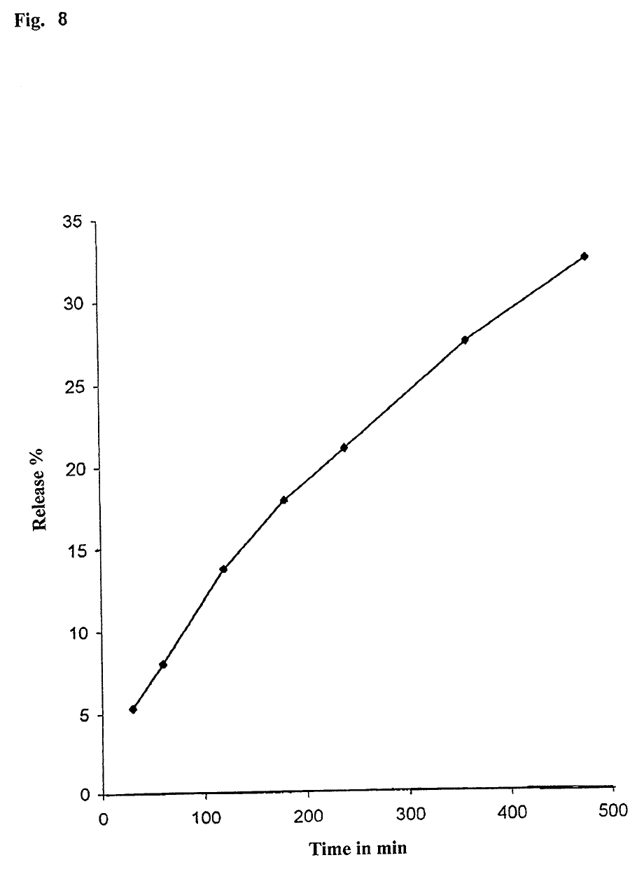

Active Ingredient Release: see FIG. 8

### EXAMPLE 11

Carbamazepine with 20% sucrose stearate of HLB 2

| Formulation: | |
|---|---|
| Substance | Amount |
| Carbamazepine | 640 g |
| Sucrose stearate S-270 | 160 g |
| Parameters: | |
| Amount of formulation | 800 g |
| Impeller speed | 700 rpm |
| Chopper speed | 3000 rpm |
| Heater jacket temp. | 54° C. |

Prepared as in Example 1

### EXAMPLE 13

Tablets from melt granulate of calcium valproate dihydrate
and 30% sucrose stearate of HLB 1

| Formulation: | |
|---|---|
| Substance | Amount |
| Calcium valproate dihydrate | 560 g |
| Sucrose stearate S-170 | 240 g |
| Parameters for granulating: | |
| Amount of formulation | 800 g |
| Impeller speed | 700 rpm |
| Chopper speed | 3000 rpm |
| Heater jacket temp. | 55° C. |

Prepared according to Example 1
Parameters for tableting:
The granulate is pressed with an oblong tableting tool,
23 mm long, 9 mm wide, into oblong tablets with a gross
weight of 951 mg and crush strength of 65 N.

Evaluation: Active Ingredient Release

| | | | |
|---|---|---|---|
| Time in min | 60 | 240 | 480 |
| Release in % in pH 3.0 | 4.96 | 9.14 | 13.66 |
| Release in % in pH 6.8 | 92.93 | 98.57 | 99.43 |

Prepared according to Example 10

### EXAMPLE 15

Retigabine with 20% sucrose stearate of HLB 1

| Formulation: | |
|---|---|
| Substance | Amount |
| Retigabine | 800 g |
| Sucrose stearate S-170 | 200 g |
| Parameters: | |
| Amount of formulation | 1000 g |
| Impeller speed | 700 rpm |
| Chopper speed | 3000 rpm |
| Heater jacket temp. | 52° C. |

Prepared according to Example 1

Evaluation: Active Ingredient Release

| | | | | | | | |
|---|---|---|---|---|---|---|---|
| Time in min | 30 | 60 | 120 | 180 | 240 | 360 | 480 |
| Release in % | 37.23 | 46.71 | 75.81 | | | | |
| in 0.1 N HCl | | | | | | | |
| In a buffer at a pH of | 5.71 | 8.77 | 13.82 | 15.79 | 23.60 | 27.99 | 35.62 |
| 6.8/1% Texapon | | | | | | | |

Active Ingredient Release: see FIG. 12

### EXAMPLE 16

Retigabine with 20% sucrose stearate of HLB 2

| Formulation: | |
|---|---|
| Substance | Amount |
| Retigabine | 400 g |
| Sucrose stearate S-270 | 100 g |
| Parameters: | |
| Amount of formulation | 500 g |
| Impeller speed | 700 rpm |
| Chopper speed | 3000 rpm |
| Heater jacket temp. | 55° C. |

Prepared according to Example 1

Evaluation: Active Ingredient Release

| | | | | | | | | |
|---|---|---|---|---|---|---|---|---|
| Time in | 15 | 30 | 60 | 120 | 180 | 240 | 360 | 480 |
| min | | | | | | | | |
| Release | 42.28 | 62.58 | 83.53 | 100.97 | | | | |
| in % in | | | | | | | | |
| 0.1 N HCl | | | | | | | | |
| Release | 11.82 | 20.77 | 34.41 | 44.94 | 52.74 | 61.63 | 66.37 | |
| in % in | | | | | | | | |
| buffer of | | | | | | | | |
| pH 7.5 | | | | | | | | |

Active Ingredient Release: see FIG. 13

### EXAMPLE17

Retigabine with 20% sucrose stearate of HLB 1 and
10% sucrose stearate of HLB 9

| Formulation: | |
|---|---|
| Substance | Amount |
| Retigabine | 210 g |
| Sucrose stearate S-170 | 60 g |
| Sucrose stearate S-970 | 30 g |
| Parameters: | |
| Amount of formulation | 300 g |
| Impeller speed | 500 rpm |
| Chopper speed | 3000 rpm |
| Heater jacket temp. | 65° C. |

Preparation as in Example 1

Evaluation: Active Ingredient Release:

| | | | | | | | | |
|---|---|---|---|---|---|---|---|---|
| Time in | 15 | 30 | 60 | 120 | 180 | 240 | 360 | 480 |
| min | | | | | | | | |
| Release | 71.49 | 85.13 | 97.93 | 102.82 | | | | |
| in % in | | | | | | | | |
| 0.1 N HCl | | | | | | | | |
| Release | 31.02 | 36.93 | 51.97 | 61.25 | 70.63 | 79.01 | 78.77 | |
| in % in | | | | | | | | |
| a buffer | | | | | | | | |
| of pH 6.8 | | | | | | | | |

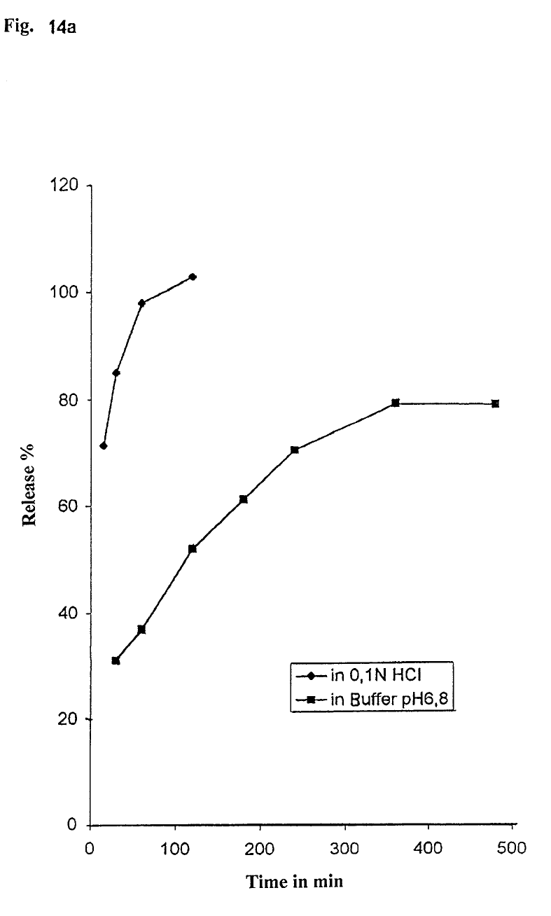

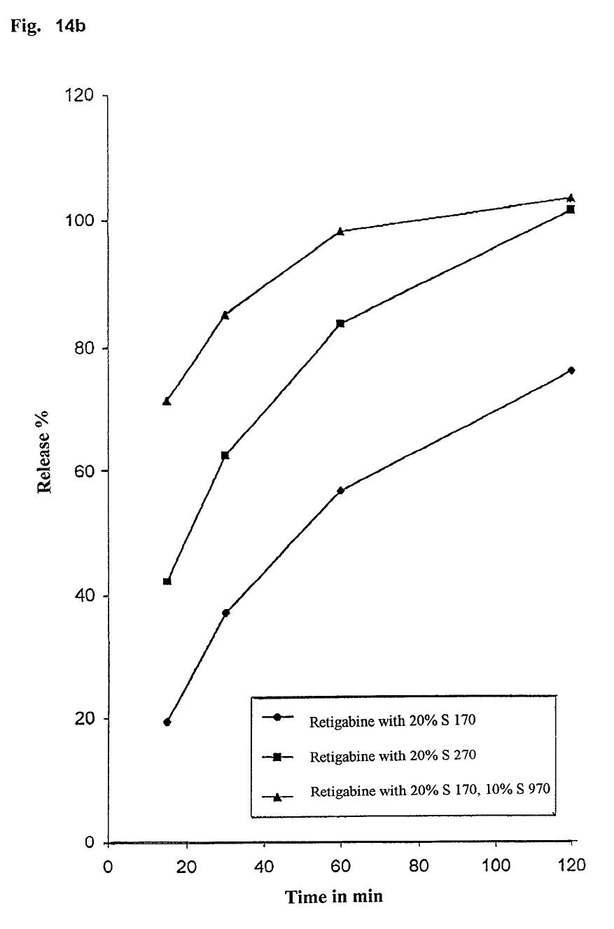

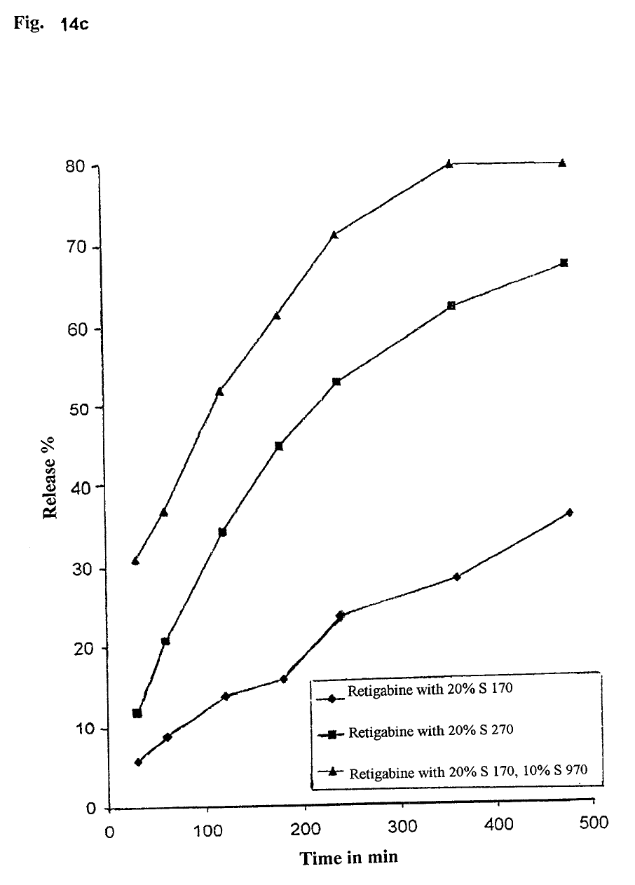

Active Ingredient Release: see FIG. 14a
For a comparison of the Active Ingredient Release from Retigabine
formulations in 0.1 N HCl, see FIG. 14b
For a comparison of the Active Ingredient Release from Retigabine
formulations in a buffer of pH 6.8, see FIG. 14c

### EXAMPLE 18

Tablets of melt granulate with retigabine, 20% sucrose
stearate of HLB 1 and 10% croscarmelose sodium

| Formulation: | |
|---|---|
| Substance | Amount |
| Retigabine | 800 g |
| Sucrose stearate S-170 | 200 g |
| Parameters for granulating: | |
| Amount of formulation | 1000 g |
| Impeller speed | 700 rpm |
| Chopper speed | 3000 rpm |
| Heater jacket temp. | 52° C. |

Prepared according to Example 1

| Parameters for tableting: | |
|---|---|
| Substance | Amount |
| Retigabine retard - granulate (see | 270 g |
| above) | |
| Croscarmellose sodium | 30 g |

The tableting mixture is pressed into tablets with a round
tableting tool with a diameter of 9 mm, a bevel of 450,
and a radius of curvature R13.

Evaluation: Active Ingredient Release

| | | | | | | | | |
|---|---|---|---|---|---|---|---|---|
| Time in | 15 | 30 | 60 | 120 | 180 | 240 | 360 | 480 |
| min | | | | | | | | |
| Release | 40.76 | 81.59 | 96.13 | 100.76 | | | | |
| in % in | | | | | | | | |
| 0.1 N HCl | | | | | | | | |
| In a buffer | 22.20 | 29.80 | 38.95 | 46.49 | 53.58 | 60.85 | 64.69 | |
| of pH 6,8/ | | | | | | | | |
| 1% sodium | | | | | | | | |
| dodecyl | | | | | | | | |
| sulfate | | | | | | | | |
| (SDS) | | | | | | | | |

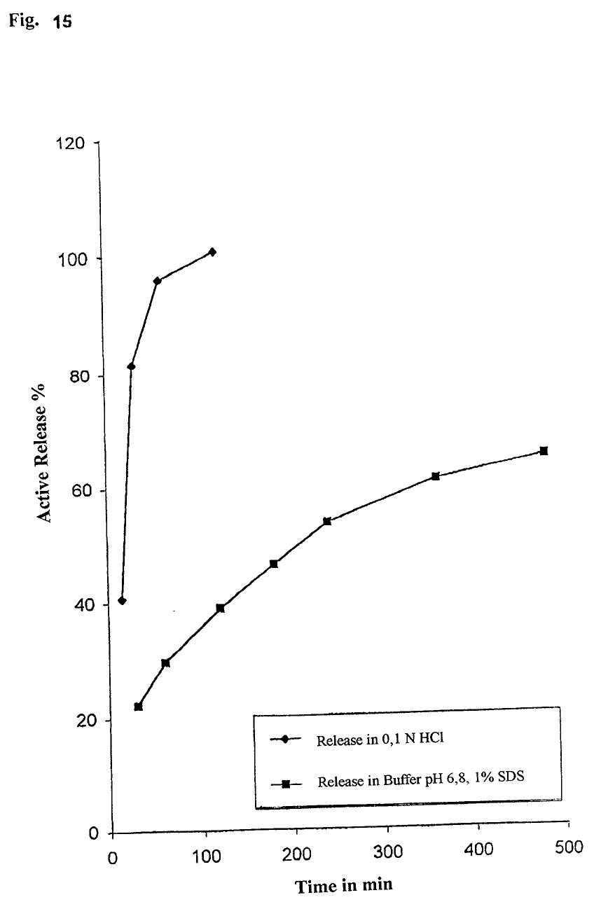

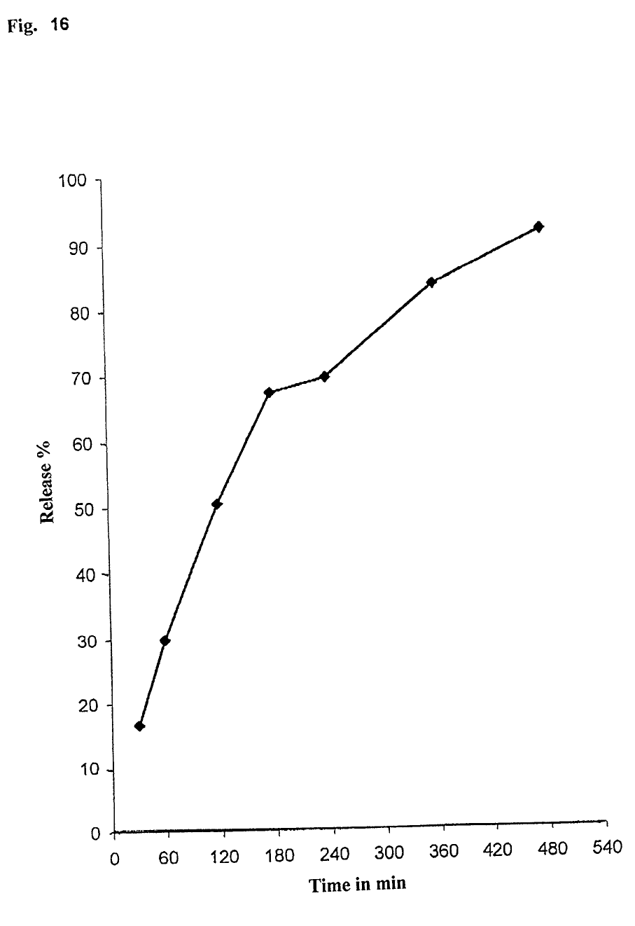

Active Ingredient Release: see FIG. 15

Example 19:
Retigabine with 7% sucrose stearate of HLB 1

| | |
|---|---|
| Substance | Amount |

Formulation:

| | |
|---|---|
| Retigabine | 372 g |
| Sucrose stearate S-170 | 28 g |

Parameters:

| | |
|---|---|
| Amount of formulation | 400 g |
| Impeller speed | 1300 rpm |
| Heater jacket temp. | 50° C. |

In an high shear mixer of the GP 1 type of firm Aeromatic-Fielder,
the starting materials are heated with stirring at a mantle temperature
of 50.0° C. in a special bowl, which is provided with a PTFE
in-liner. After the power consumption uptake has increased once again,
the pellets are removed and cooled to room temperature in thin layers.
Evaluation: Active Ingredient Release

| Time in min | 30 | 60 | 120 | 180 | 240 | 360 | 480 |
|---|---|---|---|---|---|---|---|
| Release in a buffer | 16.62 | 29.85 | 50.39 | 67.14 | 69.35 | 83.20 | 90.96 |
| of pH 7.5/2.5 | | | | | | | |
| SDS | | | | | | | |

Active Ingredient Release: see FIG. 16

### EXAMPLE 20

Retigabine with 20% sucrose stearate of HLB 11

| Formulation: | |
|---|---|
| Substance | Amount |
| Retigabine | 320 g |
| Sucrose stearate S-1170 | 80 g |
| Parameters: | |
| Amount of formulation | 400 g |
| Impeller speed | 1300-1100 rpm |
| Heater jacket temp. | 50° C. |

Prepared according to Example 19

Evaluation: Active Ingredient Release

| | | | | | |
|---|---|---|---|---|---|
| Time in min | 30 | 60 | 120 | 180 | 240 |
| Release in a buffer of | 49.91 | 79.95 | 100.81 | 106.03 | 104.36 |
| pH 7.5/2.5% SDS | | | | | |

Active Ingredient Release: see Example 17

### EXAMPLE 21

Retigabine with 20% sucrose stearate of HLB 16

| Formulation: | |
|---|---|
| Substance | Amount |
| Retigabine | 320 g |
| Sucrose stearate S-1170 | 80 g |
| Parameters: | |
| Amount of formulation | 400 g |
| Impeller speed | 1300-1100 rpm |
| Heater jacket temp. | 50-55° C. |

Prepared according to Example 19

Evaluation: Active Ingredient Release

| | | | | | |
|---|---|---|---|---|---|
| Time in min | 30 | 60 | 120 | 180 | 240 |
| Release in a buffer of | 41.77 | 68.71 | 92.32 | 99.95 | 101.47 |
| pH 7.5/2.5% SDS | | | | | |

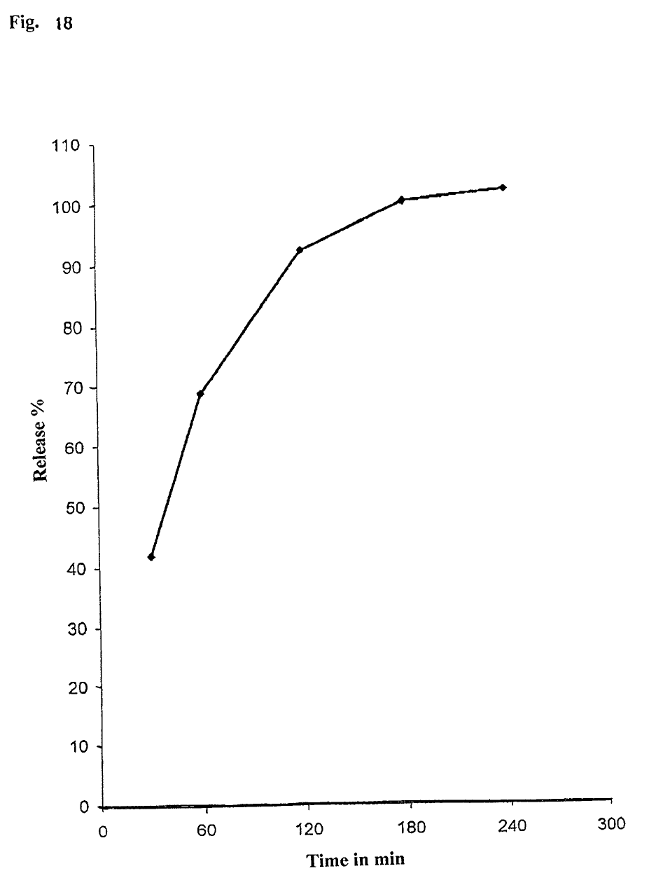

Active Ingredient Release: see FIG. 18

### EXAMPLE 22

Retigabine with 16% sucrose stearate of HLB 15

| Formulation: | |
|---|---|
| Substance | Amount |
| Retigabine | 336 g |
| Sucrose stearate S-1570 | 64 g |
| Parameters: | |
| Amount of formulation | 400 g |
| Impeller speed | 1300 rpm |
| Heater jacket temp. | 50-60° C. |

Prepared according to Example 19

Evaluation: Active Ingredient Release

| | | | | | |
|---|---|---|---|---|---|
| Time in min | 30 | 60 | 120 | 180 | 240 |
| Release in a buffer of | 64.67 | 89.83 | 99.98 | 101.78 | 100.99 |
| pH 7.5/2.5% SDS | | | | | |

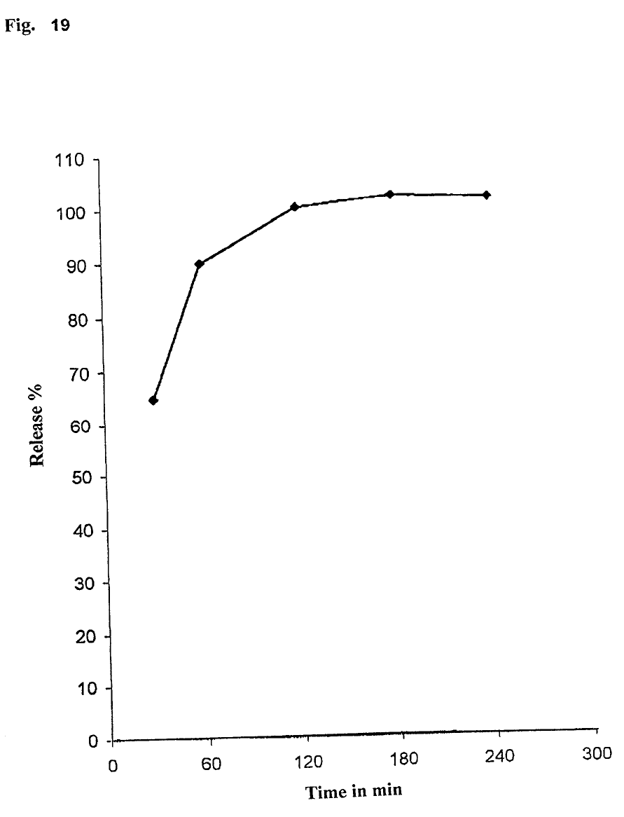

Active Ingredient Release: see FIG. 19

### EXAMPLE 23

Retigabine tablets

| Formulation melt granulate: | |
|---|---|
| Substance | Amount |
| Retigabine | 332 g |
| Sucrose stearate S-1570 | 68 g |
| Parameters: | |
| Amount of formulation | 400 g |
| Impeller speed | 1300 rpm |
| Heater jacket temp. | 50-60° C. |
| Prepared as in Example 19 | |
| Coating formulation: | |
| Substance | Amount |
| Retigabine - melt granulate with | 1000 g |
| 17% sucrose stearate of HLB 15 | |
| Eudragit L 30 D-55 | 400 g (corresponds to |
| | 120 g of lacquer |
| | solids) |
| Talcum | 60 g |
| Triethyl citrate | 12 g |

The melt granulates from 5 batches are combined and sprayed in a rotor granulator with an inflow of air at 50° C. at 300 rpm with a suspension of Eudragit L 30 D-55, talcum and triethyl citrate in 536 g of purified water. This is followed by drying up to a product temperature of 33° C.

The granulate, so coated, is homogenized for 10 minutes in a Turbula with 30% by weight of microcrystalline cellulose and 5% by weight of croscarmellose sodium.

The tableting mixture is pressed into oblong 17×8 mm, curved tablets with an average crush strength of 87 N.

| | | | | | | |
|---|---|---|---|---|---|---|
| Time in min | 15 | 30 | 60 | 120 | 180 | 240 |
| Release in % in | 50.3 | 68.8 | 83 | 88.3 | | |
| 0.1N HCl | | | | | | |
| In a buffer of pH 7.5/ | 16.1 | 30.4 | 55.7 | 83.4 | 95.2 | 99.2 |
| 1.7% SDS | | | | | | |

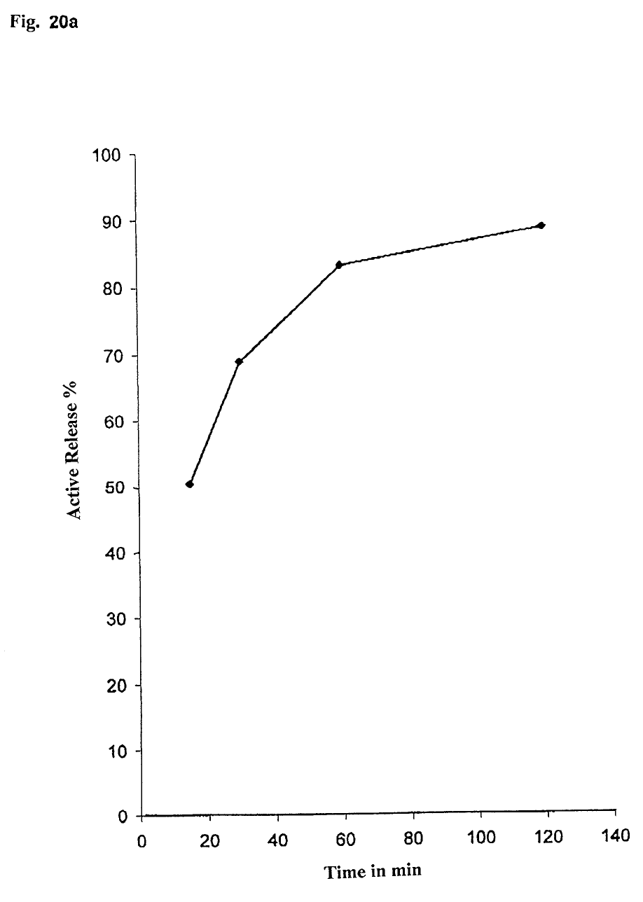

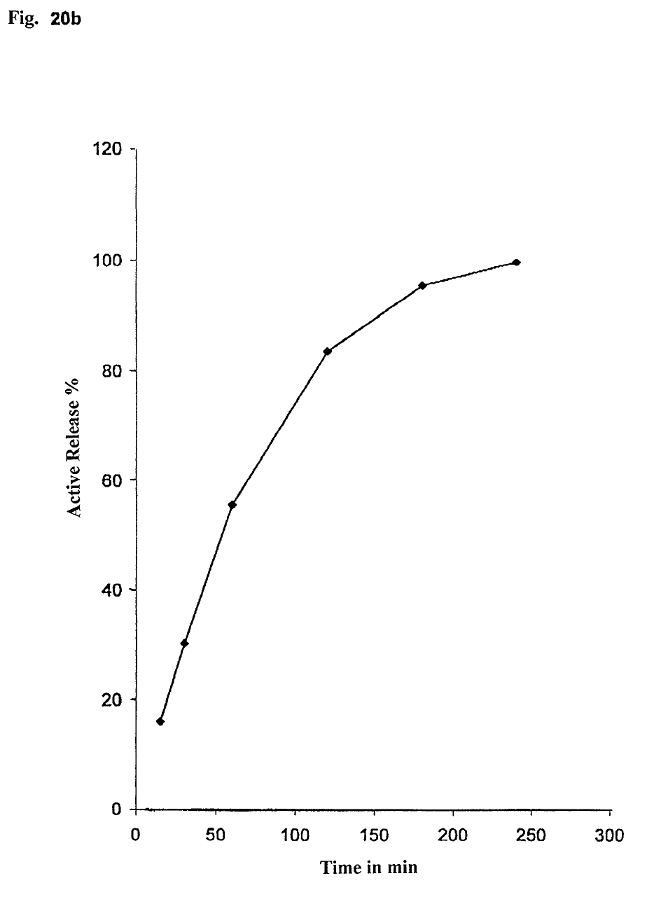

Active Ingredient Release in 0.1N HCl: see FIG. 20 a
Active Ingredient Release in a buffer of 7.5, 1.7% SDS: see FIG. 20 b

### EXAMPLE 24

Hot melt coating of retigabine melt granulate with 10%
sucrose stearate of HLB 1

| | |
|---|---|
| Substance | Amount |

Formulation of coated melt granulate:

| | |
|---|---|
| Retigabine melt granulate | 500 g |
| (90% by weight of retigabine and | |
| 10% by weight of sucrose | |
| stearate S-170) | |
| Sucrose stearate S-170 | 55.6 g |

Parameters:

| | |
|---|---|
| Amount of formulation | 555.6 g |
| Impeller speed | 700 rpm |
| Heater jacket temp. | 52° C. |

In a high shear mixer of the GP1 type of the firm Aeromatic-Fielder, the retigabine melt granulate is heated with stirring at a heater jacket temperature of 52° C. At a product temperature of 30° C., sucrose stearate S- 170 is added and granulated for a further 7 minutes with the chopper switched on (3000 rpm). The coated granulate was removed and screened through a 1.4 mm mesh screen.

Results: particle size distribution

| Particle size (μm) | Percentage (%) |
|---|---|
| >1000 | 3.4 |
| >800 | 4.0 |
| >500 | 34.4 |
| >315 | 40.9 |
| >160 | 14.0 |
| >50 | 3.4 |
| <50 | 0 |

| | | | | | | | | |
|---|---|---|---|---|---|---|---|---|
| Time in min | 15 | 30 | 60 | 120 | 180 | 240 | 360 | 480 |
| Release in % in | 23.8 | 43.6 | 71.4 | 94.4 | | | | |
| 0.1 N HCl | | | | | | | | |
| In a buffer | 7.2 | 13.2 | 19.8 | 25.4 | 31 | 40.2 | 47.4 | |
| of pH | | | | | | | | |
| 7.5/2.5% SDS | | | | | | | | |

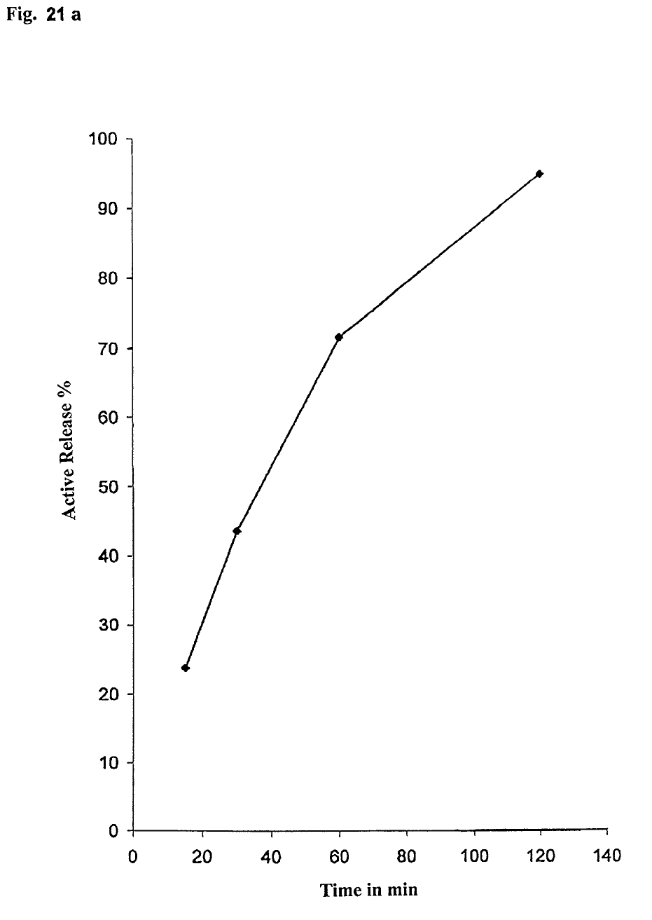

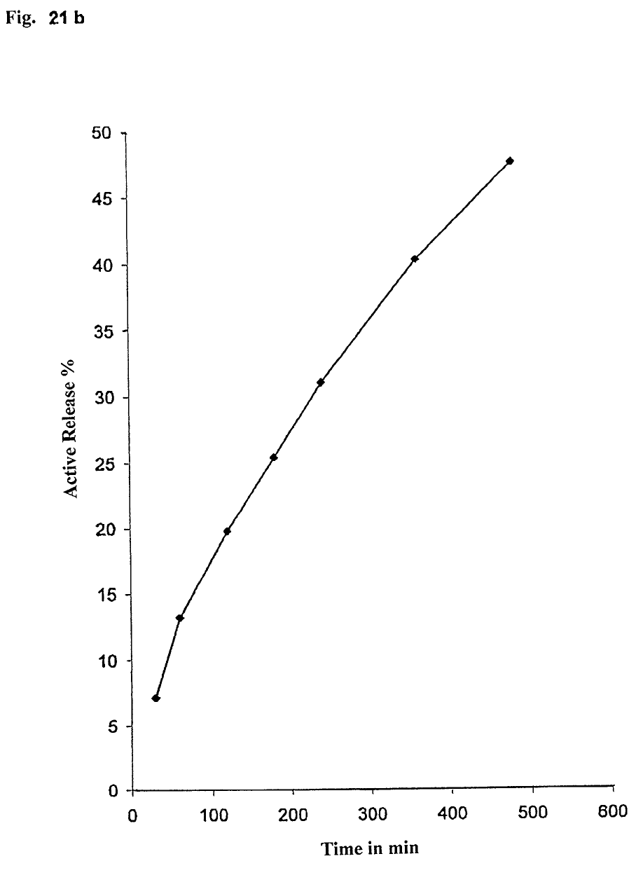

Active Ingredient Release in 0.1N HCl: see FIG. 21a
Active Ingredient Release in a buffer of pH 7.5, 2.5% SDS: see FIG. 21b

We claim:

1. An oral pharmaceutical formulation with variably adjustable release rate, which comprises one or more active ingredients, and one or more sucrose ester of a fatty acid as the sole release-controlling agent for said active ingredient.

2. The oral pharmaceutical formulation of claim 1, wherein said release rate ranges from immediate release to a predetermined controlled release.

3. The oral pharmaceutical formulation of claim 1, wherein the release rate is controlled by the type or types and concentration of said sucrose fatty acid ester, and by predetermined process parameters of the manufacturing said formulation.

4. The oral pharmaceutical formulation of claim 1, wherein said formulation is a single unit, or a multiple unit dosage form.

5. The oral pharmaceutical formulation of claims 1, having a dosage form of granules, pellets, tablets, film tablets, microtablets, sugar coated tablets, capsules, or special therapeutic dosage forms.

6. The oral pharmaceutical formulation of claim 1, wherein said active ingredient is embedded in a matrix of said sucrose ester of fatty acid, or is coated with said sucrose ester.

7. The oral pharmaceutical formulation of claim 1, having a dosage form of granules or pellets, containing a mixture of said active ingredient or active ingredients with said sucrose ester or esters, said mixture being coated with said sucrose ester or esters.

8. The oral pharmaceutical formulation of claim 1 having a dosage form of granules or pellets of said active ingredient, said granules or pellets being coated with said sucrose ester.

9. The oral pharmaceutical formulation of claim 1, wherein said sucrose ester of a fatty acid is a mono-, di-, tri- or polyesters of sucrose with a saturated and/or unsaturated fatty acid of medium to long chain length.

10. The oral pharmaceutical formulation of claim 9, wherein said fatty acid is a C₁₂ to C₂₂ fatty acid.

11. The oral pharmaceutical formulation of claim 10, wherein said fatty acid is one or more of stearic acid, palmitic acid, leuric acid, behemic acid, and oleic acid.

12. The oral pharmaceutical formulation of claim 1, wherein said sucrose ester has an HLB value of from about 1 to about 16.

13. The oral pharmaceutical formulation of claim 1, wherein said sucrose ester has a melting point of from about 30° C. to about 200° C.

14. The oral pharmaceutical formulation of claim 13, wherein said melting point is from about 40° C. to about 150° C.

15. The oral pharmaceutical formulation of claim 1, having a dosage form of a granulate, said granulate containing from about 1 to about 95% wt. of said sucrose ester.

16. The oral pharmaceutical formulation of claim 1, wherein the concentration of said sucrose ester is from about 5 to about 50% wt.

17. The oral pharmaceutical formulation of claim 8, wherein the concentration of said sucrose ester in said coating is from about 1% to about 60% wt. based on the coated formulation.

18. The oral pharmaceutical formulation of claim 17, wherein the concentration of said sucrose ester in said coating is from about 3% to about 20% wt. based on the coated formulation.

19. The oral pharmaceutical formulation of claim 1, further comprising one or more inert materials.

20. The oral pharmaceutical formulation of claim 19, wherein said inert material is one or more of a pharmaceutically acceptable filler, fusible binder, disintegrant, flow regulating agent, mold release agent, and film former.

21. An oral pharmaceutical formulation with variably adjustable release rate, which comprises one or more active ingredients, one or more sucrose ester of a fatty acid, and a pore forming agent embedded in said formulation during the forming of a dosage form thereof.

22. The oral pharmaceutical formulation of claim 1, wherein said active ingredient ranges from a water soluble material to practically water insoluble material.

23. The oral pharmaceutical formulation of claim 1, wherein said active ingredient is one or more of an analeptic agent, antihypoxemic agent, analgesic, antirheumatic agent, antiallergic agent, antiarrhythmic agent, antidementia agent, antidiabetic agent, antiemetic agent, antivertiginous agent, antiepileptic agent, antihypertensive agent, anti-hypotensive agent, broncholytic agent, antiasthmatic agent, diuretic, circulation-promoter, hypnotic agent, sedative, cardiac agent, lipid-lowering agent, antimigraine preparation, muscle relaxants, agents against extrapyramidal disorders, antiParkinson agent, and psycho-pharmaceuticals.

24. The oral pharmaceutic formulation of claim 1 which contains as active ingredient one or more of caffeine, diclofenac, morphine, tramadol, tilidine, nicargoline, pentifylline, vincamine, flupirtine, azelastine, pseudoephedrine, calcium valproate, quinidine, disopyramide, diltiazem, verapamil, piracetam, nicergolin, xantino nicotinate, pentifyllin, vincamin, glibenclamide, betahistin dimesilate, dimenhydrinate, carbamazepine, valproic acid, calcium valproat dihydrate, retigabine, talinolol, fosinopril, doxazosin, metoprolol, nifedipine, norfenefrine-HCI, dihydroergotamine mesilate, salbutamol, terbutaline sulfate, theophylline, furosemide, piretanide, buflomedil, naftidrofuryl, pentoxifylline, trinitroglycerin, isosorbide mononitrate, isosorbide dinitrate, molsidomine, bezafibrate, fenofibrate, xantinol, sumatriptan, levodopa benserazide mixture, levodopa carbidopa mixture, amitriptyline, venlafaxine-HCI, thioridazine, lithium carbonate, lithium acetate, or pharmaceutically acceptable salts thereof.

25. The oral pharmaceutical formulation of claim 24, having the dosage form of a granule of retigabine with from about 1% to about 95% of said sucrose ester in said granule.

26. The oral pharmaceutical formulation of claim 25, wherein the concentration of said sucrose ester is from about 5% to about 50%.

27. A process for preparing the oral pharmaceutical formulation of claim 1, wherein the dosage form is a granule or a pellet, which comprises melting the oral formulation, and granulating or pelletizing the melt.

28. The method of claim 27, said melting comprising heating said formulation with stirring or in a fluidized bed to a temperature at which said sucrose ester softens, commences to melt at the surface, or completely melts, forming said granules or pellets, and cooling them.

29. The method of claim 28, which comprises heating said active ingredient in powder form, and adding said sucrose ester to the heated powder.

30. The method of claim 28, wherein said heating is carried out in a high speed mixer, a high shear mixer, fluidized bed, or a rotor granulator.

31. The method of claim 27, wherein said formulation further comprises a plasticiser.

32. The method of claim 29, wherein said formulkation further comparises a plasticiser.

33. The method of claim 31, wherein said plasticiser is one or more of triethyl citrate, acetyl triethyl citrate, triacetin and dibutyl sebacate.

34. The method of claim 32, wherein said plasticiser is one or more of triethyl citrate, acetyl triethyl citrate, triacetin and dibutyl sebacate.
